(19) United States
(12) Patent Application Publication
(10) Pub. No.: US 2008/0027471 A1
(43) Pub. Date: Jan. 31, 2008

(54) CLAMP FOR ATTACHING SURGICAL AIDS TO BONE DURING A SURGICAL PROCEDURE AND SYSTEM FOR DOING THE SAME

(76) Inventor: Bernhard Hauri, Staffelbach (CH)

Correspondence Address: KNOBBE MARTENS OLSON & BEAR LLP, 2040 MAIN STREET, FOURTEENTH FLOOR, IRVINE, CA, 92614, US

(21) Appl. No.: 11/831,784

(22) Filed: Jul. 31, 2007

(30) Foreign Application Priority Data
Jul. 31, 2006 (DE) 10 2006 035 602.0

(51) Int. Cl. A61B 17/58; A61B 17/88

(52) U.S. Cl. 606/151; 606/207; 606/99

(57) ABSTRACT

A clamp for attaching surgical operating aids has at least two clamp members arranged so as to be movable relative to one another and a securing element for fixing the clamp members in at least one locked position. The clamp members have a connecting mechanism that allow the clamp members to be attached to a separate setting element actuatable for transmitting a setting force onto the clamp. A setting element, such as tongs, releasably connects to the clamp, and has a connecting mechanism connectible to the connecting mechanism of the clamp and has a spacing and/or aligning mechanism. A clamp and a setting element that is releasably joined to the clamp provide an arrangement for attachment of surgical operating aids to a bone during a surgical intervention, such as during a joint replacement surgery.

### CROSS-REFERENCE TO RELATED APPLICATIONS

The present application is based on and claims priority under 35 U.S.C. §119 to German Patent Application No. 10 2006 035 602.0, filed Jul. 31, 2006, the entire contents of which are hereby incorporated by reference and should be considered a part of this specification.

### BACKGROUND OF THE INVENTION

1. Field of the Invention

The invention relates to a clamp for temporarily attaching surgical operating aids to a bone during a surgical procedure, and to systems for doing the same.

2. Description of the Related Art

A clamp for attaching surgical operating aids is known, for example, from WO 00/00093. Such clamps are used for temporary fixing of operating aids to bones, especially long bones, in order to ensure firm positional stability between the operating aid and the bone during surgical interventions, such as the installation of joint replacement implants.

Surgical interventions for the replacement of joints or joint parts in human beings have been known for a long time and form part of everyday clinical procedure. In parallel with further development of implant structures, suitable operating techniques and aids, including tools for the installation of implants that are matched to the implant structures in question, are also being developed.

In recent years, a relatively large number of new positioning aids and methods have been developed. For example, WO 03/096870 A2, which originates from the applicant, discloses an arrangement and a method for intra-operative determination of the position of a joint replacement implant, and navigation technology used during an operation to effect continuous detection of the position of parts of the skeleton in a three-dimensional co-ordination system. In such an arrangement, a multi-camera system detects so-called multipoint indicators—also often referred to in practice as a marker field or locator—which are themselves joined in fixed position to the part of the skeleton in question. By scanning points of interest on the part of the skeleton via a measuring sensor, the scanned points are assigned to the co-ordination system represented by the marker field. FIG. 3 of WO 03/096870 A2 shows a femoral clamp from which two pins project for mounting a locator.

WO 00/00093 A1, which also originates from the applicant, describes an extensive set of instruments for the installation of a knee joint implant. The positioning aids disclosed therein are based both on the technique of screwing the aids to the bone being treated (FIG. 1) and on a clamp technique (FIG. 16), the component imparting the clamp damping force, in the form of spindle drives, forming an integral part of the clamp. The operating aids joined to the bone in that way form the basis of a multi-axis mini-robot unit, to be constructed using a quick assembly method, which has to fulfill the functions both of a measuring jig and of a cutting jig for the resection cuts.

A commonly used method of fixing clamps is a closing mechanism driven by a threaded spindle, as disclosed in WO 00/00093 A1. Such spindle drives generate the closing force of the arms of the clamp, which are movable in opposite directions towards one another. Spindle drives have the disadvantage, however, of having a relatively large overall volume. After the clamp has been closed, that is to say while the clamp is performing its actual function as a support for an operating aid during the operation, the spindle integrated in the clamp has only a holding function.

Many of the devices known hitherto for the temporary attachment of operating aids to parts of the skeleton in the context of joint replacement surgery have the following disadvantages: the structural shape and size of the device results in a substantial invasive structure in the area surrounding the operating field, and mechanical notches caused by bores introduced into the bone for screws and pins to fix the clamp to the bone increase the risk of fractures.

### SUMMARY OF THE INVENTION

In view of the circumstances noted above, an aspect of at least one of the embodiments disclosed herein is to provide a clamp for attaching surgical operating aids which can be securely joined to the selected bone, while minimizing impairment of the surrounding tissue.

In accordance with one embodiment of the invention, a clamp for attaching surgical aids to a bone is provided. The clamp comprises at least two clamp members moveable relative to each other, and a securing mechanism configured to secure the clamp members to each other in at least one locked position, wherein the clamp members comprise a connecting mechanism allowing the clamp members to be secured to a setting element actuatable to transmit a setting force onto the clamp members to move the clamp members between an unlocked position and the at least one locked position.

In accordance with another embodiment of the invention, a system for attaching surgical aids to a bone is provided. The system comprises a clamp comprising at least two clamp members moveable relative to each other, the clamp members configured to be releasably locked in at least on locked position, and a setting element removably coupleable to the clamp members, the setting element actuatable to move the clamp members relative to each other from an unlocked position to the at least one locked position.

In accordance with yet another embodiment of the invention, a method for attaching surgical aids to a bone is provided. The method comprises accessing an elongated bone of a patient, anchoring a clamp to the bone, the clamp comprising at least two clamp members that are movable relative to each other and releasably lockable in at least one locked position, and attaching a surgical aid to the clamp.

### BRIEF DESCRIPTION OF THE DRAWINGS

These and other features, aspects and advantages of the present inventions will now be described in connection with preferred embodiments, in reference to the accompanying drawings. The illustrated embodiments, however, are merely examples and are not intended to limit the inventions. The drawings include the following 13 figures.

FIG. 1 is a schematic perspective view of one embodiment of a clamp member.

FIG. 2 is a schematic perspective view another embodiment of a clamp member.

FIG. 3a is a schematic perspective view of an assembled clamp having the clamp members shown in FIGS. 1 and 2, the clamp being clamped on a bone.

FIG. 3b is a schematic perspective view of the clamp in FIG. 3a without a bone.

FIG. 4 is a schematic cross-sectional view of the clamp of FIG. 3a attached to a mounting aid or setting element.

FIG. 4a is a schematic side view of the clamp and the setting element of FIG. 4 in various positions.

FIG. 4b is a schematic perspective view of the clamp and setting element of FIG. 4 in a fully open position.

FIG. 5 is a schematic perspective view of the clamp of FIG. 3a with a surgical operating aide coupled thereto.

FIG. 6a is a schematic perspective view of a second embodiment of a clamp having a coupled securing stirrup.

FIG. 6b is a schematic perspective view of the clamp of FIG. 6a with a decoupled securing stirrup.

FIG. 7 is a schematic cross-sectional view of the clamp and mounting aid or setting element of FIG. 6a.

FIG. 7a is a schematic detailed view of the tooth-engagement region according to FIG. 7; and

FIG. 7b is a schematic perspective view of the arrangement of FIG. 7.

### DETAILED DESCRIPTION OF THE PREFERRED EMBODIMENTS

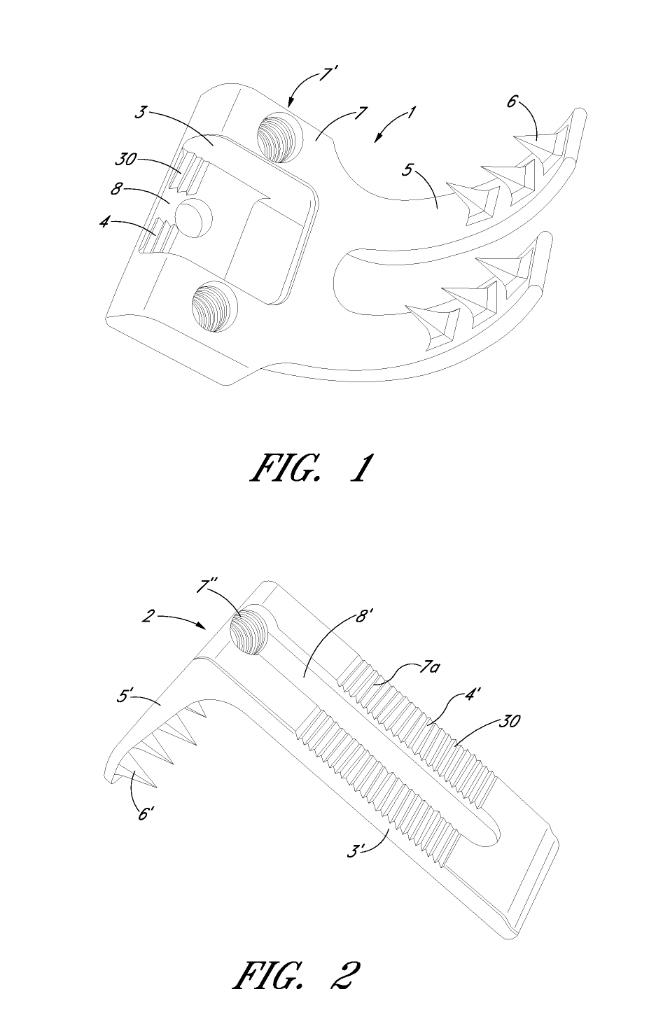

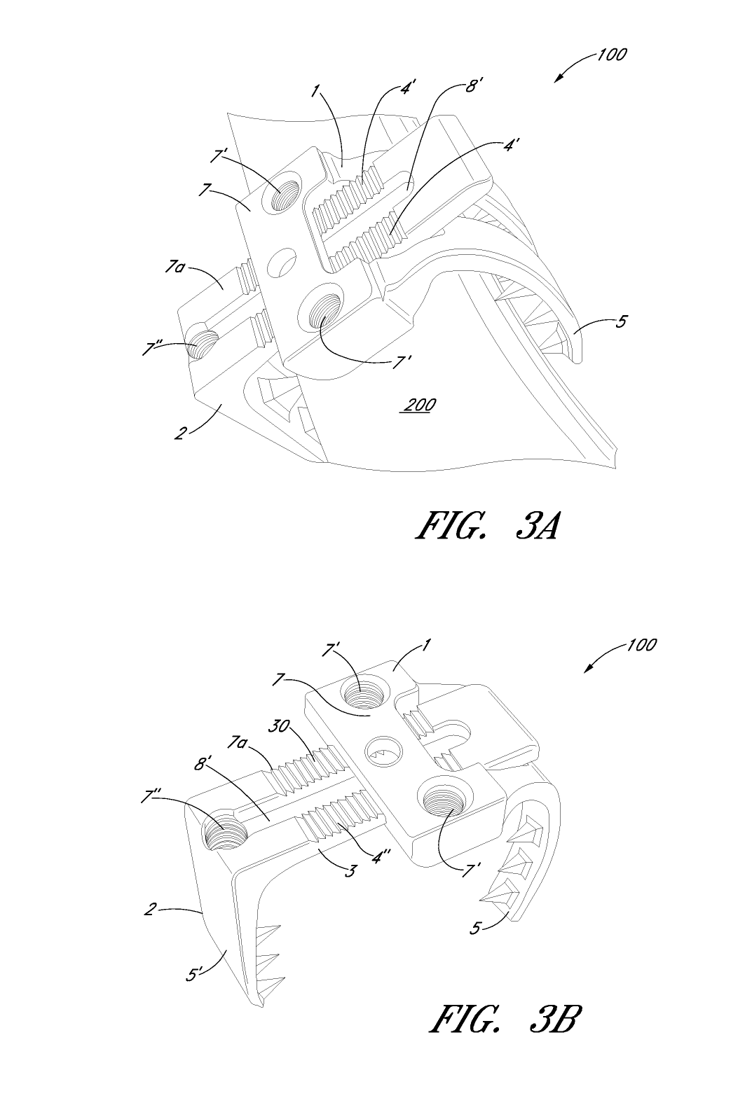

FIGS. 1-4b show one embodiment of a bone clamp 100, which can have a first clamp half or clamp member 1 (see FIG. 1) and a second clamp half or clamp member 2 (see FIG. 2). However, the clamp 100 can have more than two clamp members movable relative to one another, especially in opposite directions, to generate a clamping force, and is not limited to clamps consisting of two clamp members or two clamp halves.

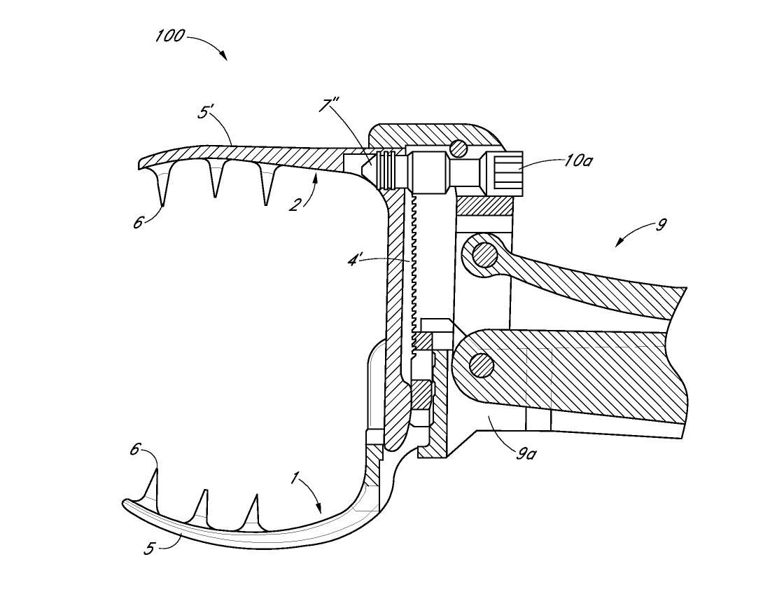

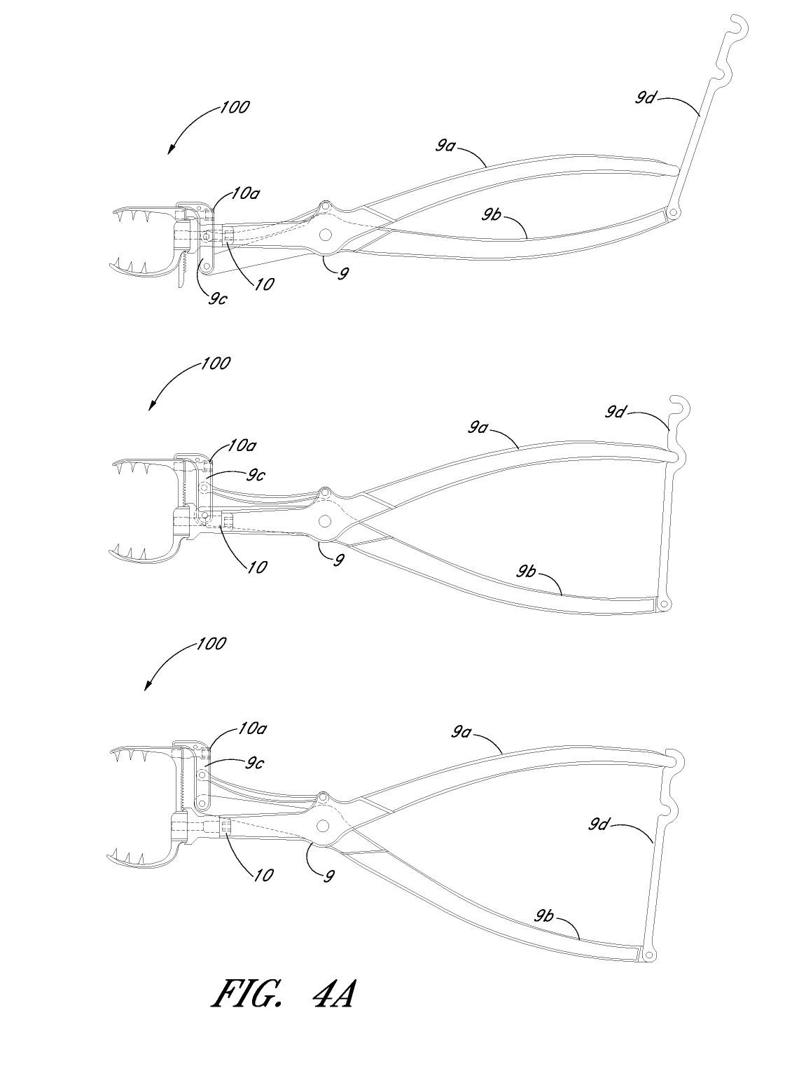

The first clamp member 1 can have a guide/adaption part 7 with two clamp arms 5, each of the arms 5 having anchoring elements 6 thereon. In one embodiment, the anchoring elements 6 can be removably attached to the arms 5. In another embodiment, the anchoring elements 6 can be formed on the arms 5. The clamp 100 can therefore be anchored stably on a bone 200 (see FIG. 3a). The second clamp member 2 can also have a guide/adaption part 7a with a clamp arm 5′ which can be arranged opposite the clamp arms 5 of the first clamp member 1 when the clamp 100 is in use (see e.g., FIG. 3b). When the clamp 100 is in the assembled configuration (see FIG. 3a), the adaption part 7 of the first clamp member 1 can extend substantially transversely with respect to the adaption part 7a of the second clamp member 2.

In the illustrated embodiment, the clamp 100 has a guide mechanism formed on the two clamp members 1, 2, which can be a linear guide for facilitating coupling of the clamp members 1, 2 to assemble the clamp 100. In the illustrated embodiment, the guide mechanism includes an internal guide 3 formed on the first clamp member 1 and external guide 3′ formed on the second clamp member 2. The internal guide 3 can be a slot or recess formed on the guide/adaptation part 7. The external guide 3′ can be outer edges of the second clamp member 2 that can slide within the internal guide 3 so that the edges of the second clamp member 2 are positioned between opposing walls of the slot or recess. However, the guide mechanism can have other suitable configurations that allow relative movement and engagement of the clamp members 1, 2, and is not limited to the linear guide in the illustrated embodiment. For example, articulated connections, such as pivot joints, can also be used.

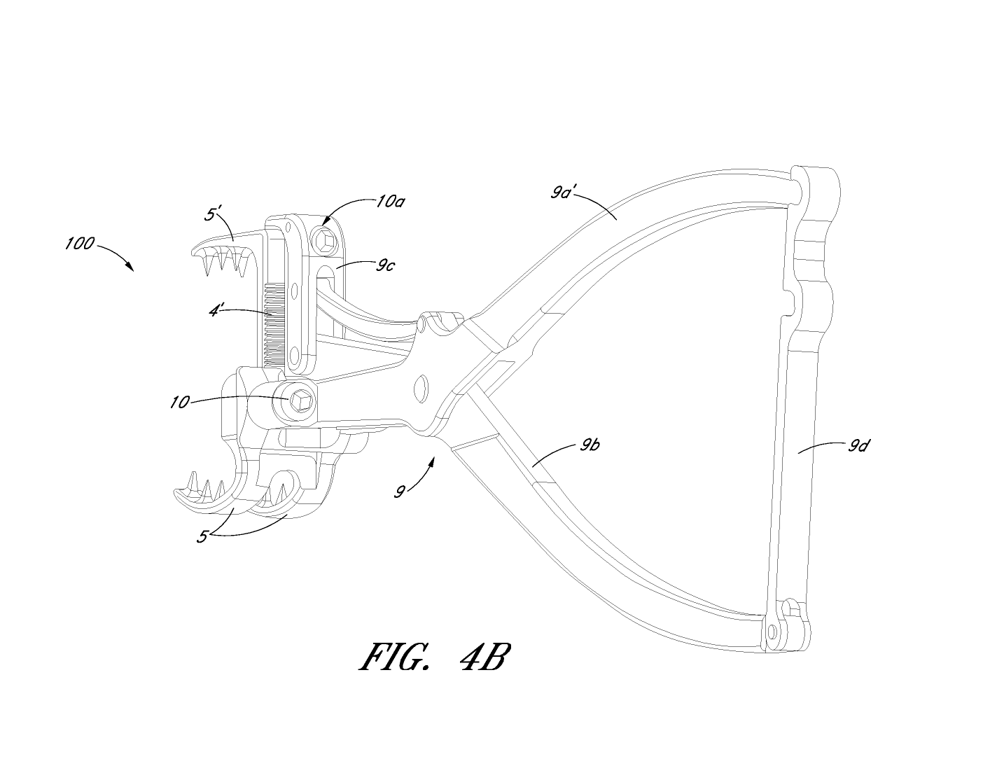

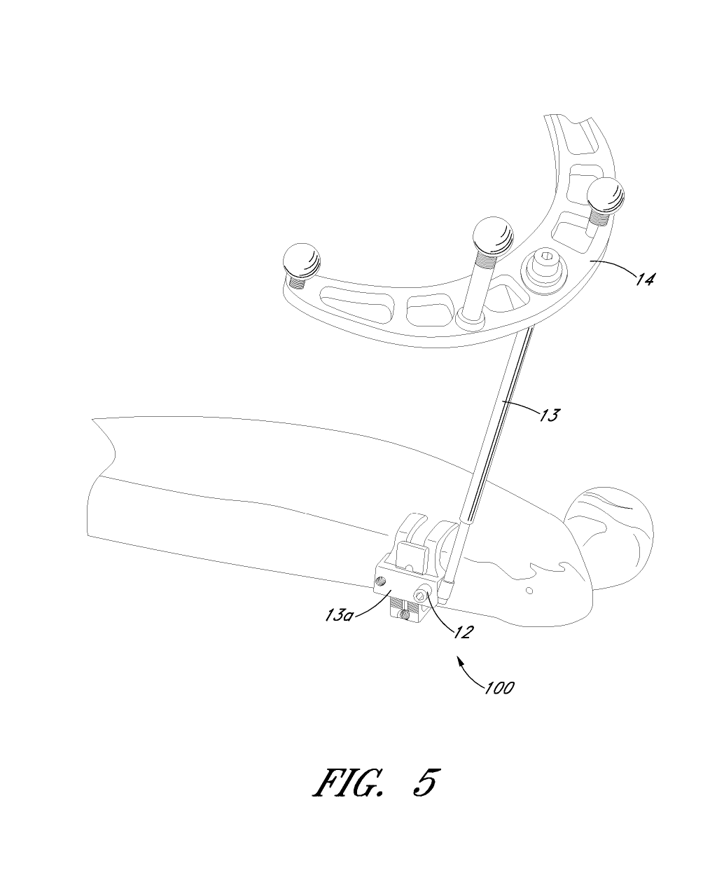

A mounting aid 9 (see FIGS. 4-4b), which is discussed further below, limits any degree of freedom to tilt that exists in the clamp 100 arrangement in the first coupling phase of the clamp members 1, 2 (e.g., when the clamp members 1, 2 are initially slid-together), where in one embodiment the mounting aid 9 guides the clamp members 1, 2 in parallel. Additionally, any degree of freedom to tilt that exists in the clamp 100 is inhibited once a clamping reaction force of the bone 200 is exerted on the clamp arms 5, 5′.

With continued reference to FIGS. 1-4b, the clamp 100 can have a securing mechanism 30 with interlocking elements, or engagement elements, to counter unintentional opening of the clamp 100 once it has been mounted to the bone 200. In the illustrated embodiment, the engagement elements are a plurality of teeth 4 formed on the first clamp member 1 that can engage teeth 4′ formed on the second clamp member 2 to substantially fix the clamp members 1, 2 relative to each other in at least one position. The teeth 4, 4′ can be formed on surfaces of the clamp members 1, 2, respectively, that are arranged to oppose each other during assembly of the clamp 100. In the illustrated embodiment, the teeth 4, 4′ have a saw-tooth shape, but can have other suitable shapes. Additionally, the interlocking or engagement elements can have other suitable configurations and are not limited to the teeth 4, 4′ illustrated in the embodiment of FIGS. 1-4b. For example, locking catches or other interlocking elements can also be used.

As shown in FIG. 2, the teeth 4′ can be arranged over a relatively long portion of the length of the second clamp member 2. In another embodiment, the teeth 4′ can be formed on substantially the entire length of the second clamp member 2. The teeth 4 of the first clamp member 1 can be arranged over a short portion of its length, such that the length of the teeth 4 portion is shorter than that of the teeth portion 4′. In the illustrated embodiment, the teeth 4 are formed on a base of the internal guide 3 or recess, and the teeth 4′ are formed on a rear surface of the second clamp member 2. However, the teeth 4, 4′ can be formed on other surfaces of the clamp members 1, 2 that allow for relative motion and locking engagement of the clamp members 1, 2. In one embodiment (not shown), the teeth 4, 4′ can form part of the guide mechanism, with teeth formed on the side walls of the recess of the first clamp member 1, and corresponding teeth formed on the outer edges of the second clamp member 2.

In one embodiment, the teeth 4, 4′ engage each other at the latest when the reaction force of the bone 200 starts to act on the clamp 100. The reaction force of the bone 200 can cause the teeth 4, 4′ to be pressed against the base of the tooth gaps of the opposing teeth and so the two clamp members 1, 2 can be held in secure interlocking engagement with one another. The teeth 4, 4′ can have different configurations (e.g., of tooth pitch) to provide clamps that provide relatively hard closing or clamps that provide relatively soft closing. The pitch of the teeth 4, 4′ can also be varied to secure against the effects of extreme force and vibrations.

As shown in FIGS. 1 and 2, the clamp members 1, 2 can each have a connecting mechanism through which the clamp members 1, 2 can releasably couple to the separate mounting aid or setting element 9. As shown in the illustrated embodiment, the connecting mechanism can be threaded bores 7′, 7″ formed on the adaption parts 7, 7′ of the clamp members 1, 2, respectively, that are dimensioned for receiving suitable screws, which can be mounted in the separate setting element 9.

In the illustrated embodiment, the connecting mechanism includes three threaded bores 7′, 7″. Two of the threaded bores 7′ are provided in the adaption part 7, which extends generally transversely to the adaptation part 7a, so that the bores 7′ are close to the outer edges of the adaptation part 7a. The third threaded bore 7″ is provided in the adaptation part 7a of the second clamp member 2, close to the edge where the clamp arm 5′ joins to the adaptation part 7a. The threaded bore 7″ of the other (second) clamp member 2 is disposed generally in the middle between the two other threaded bores 7′ in the assembled clamp 100. Accordingly, the connecting mechanism (e.g., threaded bores 7′, 7″) can have a symmetrical arrangement that inhibits tilting of the clamp members 1, 2 when the setting element 9 is coupled to the connecting mechanism 7′, 7″ and is actuated for transmission of a setting force to the clamp 100.

However, the connecting mechanism can have other suitable configurations and arrangements that enable the clamp members 1, 2 to be releasably joined to the setting element 9 in order to transmit a setting force from the setting element 9 to the clamp members 1, 2. The connecting mechanism is not limited to the screw connections of the illustrated embodiment. For example, force-based or interlocking connections can also be used.

The clamp 100 can be mounted on the bone 200 using the mounting aid or setting element 9, which can have arm members 9a, 9b, which can be coupled to each other in a scissor-like manner, as shown in FIGS. 4a-4b. The clamp members 1, 2 can be mounted on the mounting aid 9. An arm member 9a can have on its front face and adaption interface, which can interlock with the adaption part 7 of the first clamp member 1 (see FIG. 4), and at least one screw 10 for securely joining the first clamp member 1 to the mounting aid 9. Another arm member 9b can have on its front face a parallelogram-guided angular extension arm 9c, which can engage the second clamp member 2 (e.g. on the outside of the clamp member 2). A screw 10a can be used for holding the second clamp member 2 with a certain degree of freedom on the mounting aid 9.

In one embodiment, the clamp members 1, 2 can first be mounted on the mounting aid 9. The arms 9a, 9b of the mounting aid 9 can be fixed relative to each other by a pivotable snap-lock clip 9d that removably engages the ends of the arms 9a, 9b (e.g., the mounting aid 9 can be fixed in two-dimensional positions). As shown in FIG. 4a, the pivotable snap-lock clip 9d can be used to fix the arms 9a, 9b of the mounting aid, and thereby fix the relative distance between the clamp arms 5, 5′ from each other, in at least two positions. In one embodiment, the distance between the clamp arms 5, 5′ can be about 40 mm. In another embodiment, the distance between the clamp arms 5, 5′ can be about 33 mm. In still another embodiment, the distance between the clamp arms 5, 5′ can be about 24 mm.

At the beginning of the mounting operation, the surgeon can guide the mounting aid 9 with the arms 9a, 9b fixed by the clip 9d in one position (see FIG. 4a) towards the desired location on the bone. After straddling the bone with the clamp 100 in a desired location, the surgeon can then release the snap-lock clip 9d and actuate the closing operation of the bone clamp 100 by applying a force, such as a manual force, to the arms 9a, 9b. As soon as a reaction force of the bone is exerted on the clamp arms 5, 5′, the backs of the teeth 4 come into contact with the backs of the teeth 4′. Continuation of the closing operation results in increasing resistance from the bone, and clamp engagement increasingly involves tooth engagement combinations that are associated with greater clamping force. The closing operation can be continued until the desired clamping force of the bone clamp 100 is achieved. The mounting aid 9 can be decoupled from the clamp 100 by removing the screws 10, 10a.

To release the bone clamp 100 from engagement with the bone, an opening force, counter to the resilient tension status of the clamp members 1, 2, can be applied between an opening track 8′ and a clamp region 8 of the clamp members 1, 2, respectively, by a tool (e.g., a rocking lever) until the engaged teeth 4, 4′ become disengaged. The clamp members 1, 2 can thereby be decoupled from one another and can be removed from engagement with the bone.

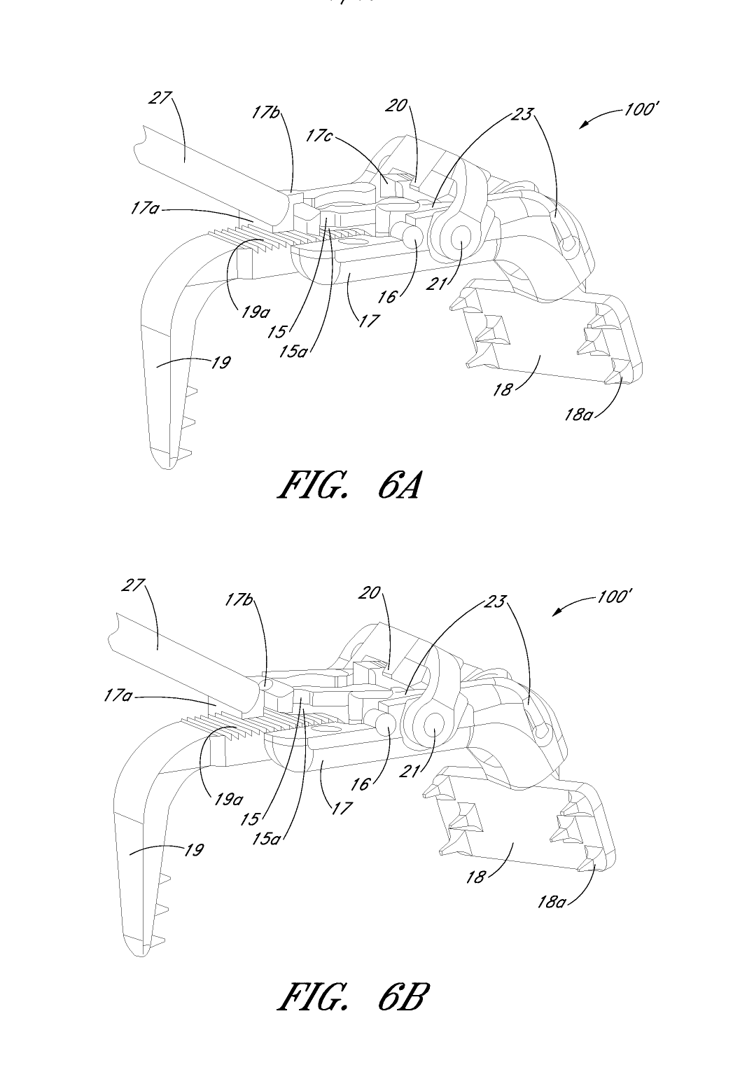

FIG. 5 shows a marker field 14 coupled to a support rod 13. The adapter-side end region 13a of the support clamp 13 is attached to the bone clamp 100 via a connecting element 12, the clamp 100 being fixed to a bone as described in the embodiments herein.

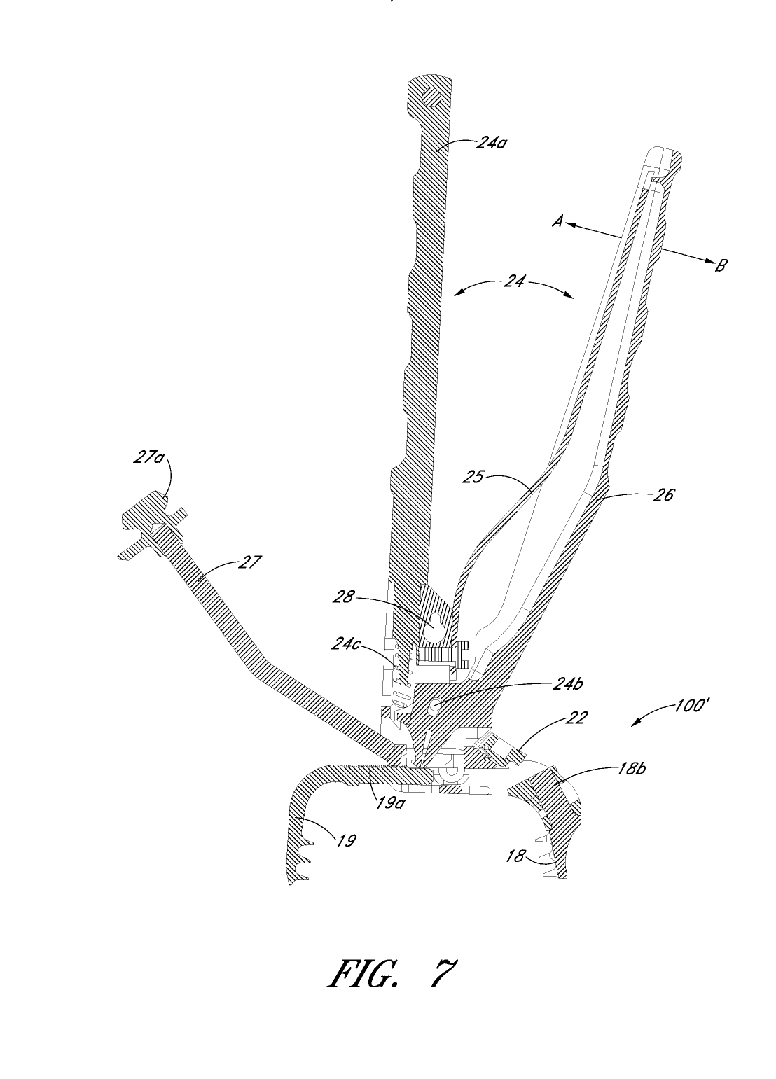

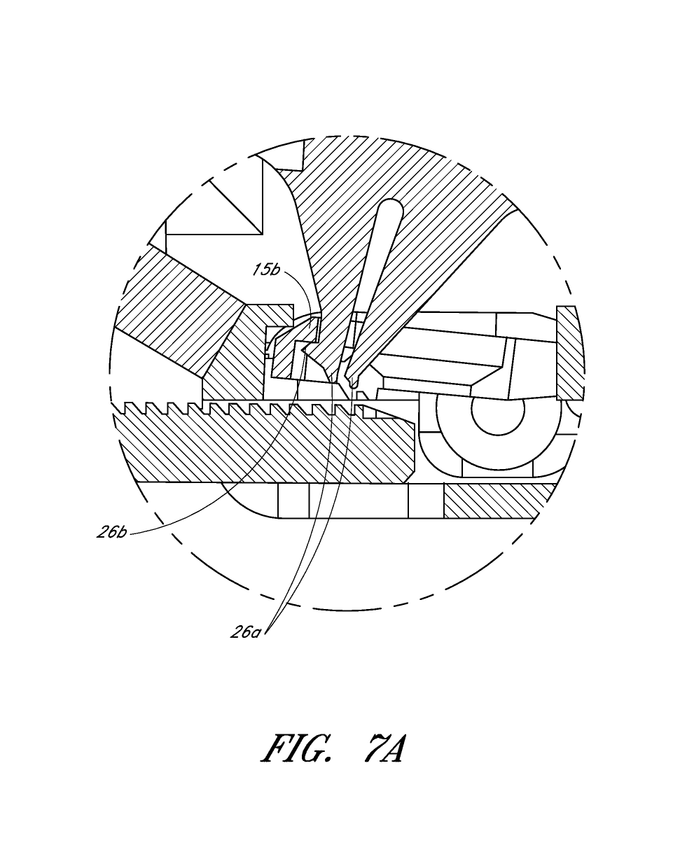

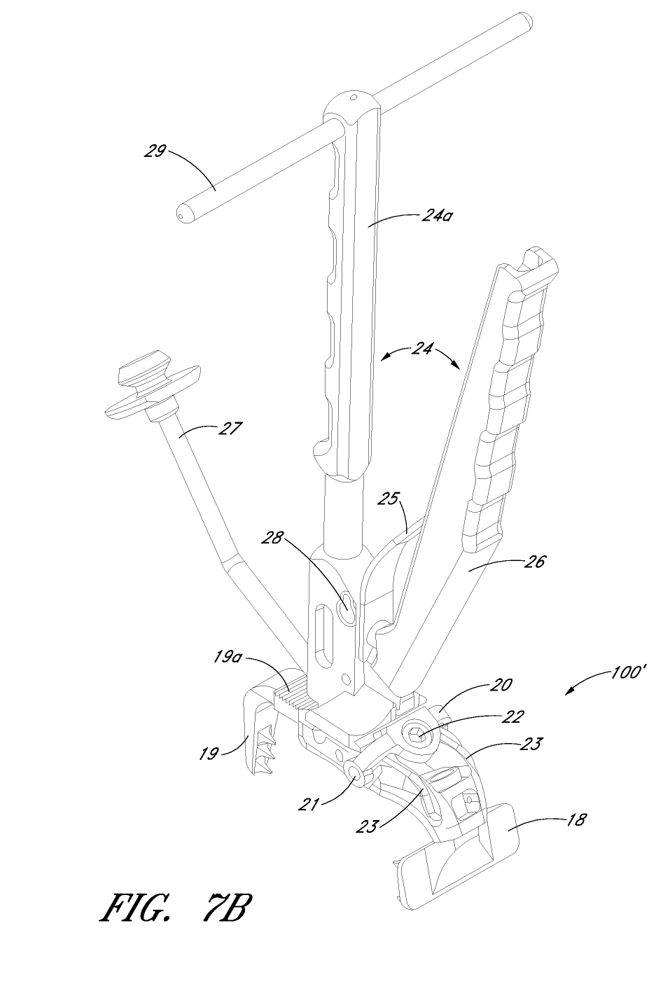

FIGS. 6a-7b show a second embodiment of a bone clamp 100′. In one embodiment, the clamp 100′ can be used for knee-joint implant operation. The clamp 100′ can have, in addition to an adaption interface 17b, 17c for mounting operating aids, such as a positioning mechanism and saw guides, a support rod 27, which can be directly integrated in the first clamp member 17 having an adaption interface 27a for coupling a locator field thereto. The direct integration of the support rod 27 can be implemented in such a way that its fixed connection to the first clamp member 17 does not interfere at any time during the operation. In another embodiment, the support rod 27 can be coupled to the first clamp member 17 by a second adaption interface (not shown) on the first bone clamp member 17.

The term “adaption interface,” as used herein, means a connecting interface arranged so as to enable the two clamp members 17, 19 to be joined to a separate setting element 24 or mounting aid. Such a connecting interface can be, for example, in the form of threaded bores or other connecting elements provided that the transmission of a setting force sufficient to attach the clamp 100′ to the bone is facilitated.

The second bone clamp member 19 (FIG. 6a) is similar to the clamp member 2 in the embodiment described above with respect to FIGS. 1-4b. The first bone clamp member 17 can have a securing stirrup 15, which can be pivotally mounted thereto by, for example, journals 16, the toothed region 15a of the securing stirrup 15 being pressed by a spring element 23 onto the opposing teeth 19a. In FIG. 6b, the securing stirrup 15 is shown in a pivoted-out position, which allows the second clamp member 19 to move freely in the guide direction within the internal guide of the first clamp member 17. The way in which that pivoted-out position is reached against the force of the spring can be seen from the description of the operation of the mounting aid or setting element 24, below.

Part of the internal guide of the first bone clamp member 17 can be provided by a support rib 17a in cooperation with further internal guide shaping of the first clamp member 17. In order to manage relatively large anatomical differences in bone shapes, the arm end 18 with its anchoring elements 18a is in the form of a component pivotally guided by pivot bearings 18b in a bearing bore, corresponding to the pivot bearing, in the first clamp member 17.

The upper end region of the first clamp member 17 and especially the shaped elements 17b, 17c serve as adaption elements both for mounting the mounting aid 24 and for receiving operating aids. For the actual fixing of the mounting aid 24 or operating aids on the first clamp member 17, there is used a retainer 20 which can be mounted on the first clamp member 17 via pivot bearings 21 and by a pressure screw 22, and can be moved out of the clamp member 17 into a pivoted position so that in the event of repeated mounting operations the adaption regions of aids to be mounted can be anchored in a fixed position in exactly the same location on the first clamp member 17.

The mounting aid 24 can have a grip region 24a having an adaption interface corresponding to the upper end region of the first clamp member 17 or the adaption interfaces 17b, 17c thereof and can have in its upper region the grip region 24a and in the central portion a bearing 24b which, in co-operation with a recess as guide region, can hold the grip member 26.

The grip member 26 can be held by a spring element 25 in a desired central position rotated about the bearing 24b and can be supported in the direction toward the second clamp member 19 under the action of gravity or a spring element 24c inside its elongate bore, which can be arranged around the bearing 24b. In said support region, the grip member 26 can have opposing tooth elements 26a which, in engagement with the row of teeth 19a, on actuation of the grip member 26 in direction of arrow A, bring about closure of the clamp 100′ by one or more tooth positions between the second clamp member 19 and the securing stirrup 15. When the grip member 26 is released, the securing stirrup 15 can hold the second clamp member 19 in its current securing position and the spring element 25 can bring the grip member 26 back into its central position, the backs of the teeth 26a sliding over the backs of teeth 19a. This operation can be repeated until the clamp 100′ is attached to the bone with a desired clamping force.

For safeguarding against overloading of the clamp 100′ it is possible, as an alternative, to provide an overload protection device, such as, for example, a frictional coupling at a suitable location, such as, for example, in the arm region of the grip member 26. If the clamp 100′ should have to be released for any reason, provided the mounting aid 24 is still fixed on the clamp member 17, the grip member 26 can be operated in direction of movement B to initiate a release function so that the hook 26b engages in an undercut 15b of the securing stirrup 15, lifts the securing stirrup 15 out of tooth engagement 15a, 19a and accordingly releases the clamp tension again (see FIG. 7a).

When the mounting aid 24 is not mounted to the clamp 100′, a tilting movement can be performed through a side opening in the first clamp member 17 using a tool, the movement tilting the securing stirrup 15 away against the resilient force of the spring 23 (analogously to the way shown in FIG. 6b) and thus releases the clamp securing mechanism.

In the case of the operating aids used in accordance with that configuration, the lowermost region of the adaption interface is arranged in a somewhat lower position relative to the interface of the mounting aid 24 and is located immediately above the securing stirrup 15 in its securing position, so that there is no room for the securing stirrup 15 to be tilted away. Accordingly, with the operating aid in place, protection is provided against inadvertent release of the clamp securing mechanism.

Although the operating aid can have a number of fine-positioning devices, it is nevertheless advisable, for the purpose of a certain rough pre-positioning, for the clamp 100′ or especially the mounting aid 24 to have devices via which the surgeon can move the clamp position on the bone closer to an ideal position.

An elongate component 29 can be attached to the grip region 24a of the mounting aid 24. In addition to providing a grip, the elongate component 29 can also fulfill a target line function in that the longitudinal axis of component 29 can be aligned approximately in parallel with the load axis of the leg (FIG. 7b). Furthermore, the mounting aid 24 can have a bayonet opening 28 for coupling-on a rough spacing jig. It is thus possible to derive the distance between the notch point of the knee and the clamp 100′ to be positioned on the bone (FIG. 7).

In certain embodiments disclosed herein, the clamp can be mounted on the bone by a removable mounting aid or setting element, which transfers a clamping or setting force to the clamp members of the clamp. Once the clamp has been mounted at the desired location on the bone, the setting element can be removed, leaving the clamp locked in place and held in the desired position by the securing mechanism thereof, the bone in question being engaged, at least in some regions, by the clamp. The clamp itself therefore needs to fulfill only the securing function and does not, as in the prior art hitherto, have a mechanism for clamping the clamp members (e.g., a spindle drive). Once the clamp has been mounted on the bone, the functions it has to perform during the further course of the surgical operation are therefore kept to a minimum. Accordingly, the size and complexity of the clamp can advantageously be reduced. The reduced size and structural shape of the clamp is also advantageous because it minimizes the disruption of muscle and tendon tracts that surround bones, especially in the region close to the joint, which have important mechanical functions.

Additionally, an important criterion for successful implantation of a joint endoprosthesis is the correct tension of the joint-stabilizing soft tissue structures. This tension can be adversely affected by large structural shapes, especially if the structure has projecting elements, so that the mechanical conditions, such as, for example, the tension of the soft tissues that bridge joints, are changed. The compact structural shape of the clamp advantageously results in substantially the same mechanical conditions before and after removal of the clamp.

In at least one of the embodiments disclosed herein, the reduction in the overall volume of the clamp is achieved at least in part by using an external, e.g., removable, element that couples to the clamp and produces a closing force thereon, wherein the external element (e.g., setting element) can be removed once the clamp has been set in place. The holding function of the clamp is fulfilled by the securing mechanism, which can have engagement elements, such as teeth, which can be associated with the clamp members and can be releasably engaged together in the locked position. As a result, the mounting tension, or clamping tension, can be retained in a simple manner.

A further advantage of the compact structural shape of the clamp is that in the case of implants having stem-like components that are to be positioned in a long bone, collisions between the attachment elements of operating aids and the instruments for preparing the stem anchoring, or the anchoring stems themselves, are avoided.

In certain embodiments, the connecting mechanism for the separate setting element can be arranged for attachment of the operating aid. Accordingly, the connecting mechanism can be joined both to the setting element and, after removal of the setting element, to an operating aid. By virtue of the overlapping of functions, the overall volume of the clamp can advantageously be further reduced. The connecting mechanisms both on the clamp and on the setting element are simple, easy to use and reliable in operation and, particularly with respect of the clamp, do not have a bulky or projecting structure. The connecting mechanism can also be constructed so that an operating aid is always brought into exactly the same position relative to the bone despite repeated coupling operations.

In certain embodiments, the clamp members of the clamp can be joined by a linear guide mechanism or by a pivot joint. In general, the guide mechanism of the clamp members is constructed so that relative movement of the clamp members for setting the desired clamping dimensions (e.g., distance between the clamp members), or sliding them one into the other, will bring about a reaction force in the clamped bone that negates the clamp members′ degree of freedom to tilt relative to one another that exists before the clamping dimensions are reached. The linear guide mechanism or the pivot joint accordingly offers a simple way of bringing the two clamp members into the holding position required for clamping the bone.

Another advantage of the embodiments disclosed herein is that a release element can be provided to co-operate with the securing mechanism to release the clamp members from the locked position. Accordingly, the fixed clamp can easily be released from the bone. It is also possible to arrange the securing mechanism so as to be movable out of the locked position into the free position and vice versa. In that way too, freeing of the bone is facilitated.

The securing mechanism can be acted upon by a restoring force which moves the securing mechanism into the locked position and/or holds it in the locked position. The security of the clamp connection is thus increased, so that reproducible attachment of the operating aids in the desired position is ensured.

In certain embodiments, the setting element can also be provided with spacing and/or aligning mechanism that advantageously simplifies handling of the setting element.

The clamps 100, 100′ disclosed herein can be made of any suitable material for use in surgical procedures to clamp to a bone. For example, the clamps can be made of a biocompatible metal or plastic material.

Although these inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. For example, though certain materials have been identified in the preferred embodiments disclosed above, one of ordinary skill in the art will recognize that other suitable materials can also be used. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above.

What is claimed is:

1. A clamp for attaching surgical aids to a bone, comprising:
at least two clamp members moveable relative to each other; and
a securing mechanism configured to secure the clamp members to each other in at least one locked position,
wherein the clamp members comprise a connecting mechanism allowing the clamp members to be secured to a setting element actuatable to transmit a setting force onto the clamp members to move the clamp members between an unlocked position and the at least one locked position.

2. The clamp of claim 1, wherein clamp members are axially moveable relative to each other.

3. The clamp of claim 1, wherein the clamp members are pivotally moveable relative to each other.

4. The clamp of claim 1, wherein the clamp members are releasably lockable to a bone via said setting element.

5. The clamp of claim 1, wherein the clamp members comprise linear guides configured to align the clamp members together to form the clamp.

6. The clamp of claim 1, wherein the securing mechanism comprises teeth on opposing surfaces of the clamp members, the teeth releasably engageable with each other to secure the clamp members to each other.

7. The clamp of claim 1, further comprising a release element configured to co-operate with the securing mechanism to release the clamp members from the at least one locked position.

8. The clamp of claim 1, wherein the clamp members comprise anchoring elements configured to anchor the clamp members to a bone.

9. The clamp of claim 1, wherein the connecting mechanism comprises at least one screw coupling the clamp members to the setting element.

10. A system for attaching surgical aids to a bone, comprising:
a clamp comprising at least two clamp members moveable relative to each other, the clamp members configured to be releasably locked in at least on locked position; and
a setting element removably coupleable to the clamp members, the setting element actuatable to move the clamp members relative to each other from an unlocked position to the at least one locked position.

11. The system of claim 10, wherein the setting element comprises a pair of arms, the arms coupled to each other in scissor-like manner, the arms coupleable to the clamp members.

12. The system of claim 10, wherein the setting element is actuatable to axially move the clamp members relative to each other.

13. The system of claim 10, wherein the setting element is actuatable to pivotally move the clamp members relative to each other.

14. The system of claim 10, wherein the clamp members are releasably lockable to each other in the at least one locked position via corresponding teeth on the clamp members, the teeth engageable with each other to lock the clamp members relative to each other.

15. A method for attaching surgical aids to a bone, comprising:
accessing an elongated bone of a patient;
anchoring a clamp to the bone, the clamp comprising at least two clamp members that are movable relative to each other and releasably lockable in at least one locked position; and
attaching a surgical aid to the clamp.

16. The method of claim 15, wherein anchoring the clamp comprises pivoting the clamp members relative to each other.

17. The method of claim 15, wherein anchoring the clamp comprises axially moving the clamp members relative to each other.

18. The method of claim 15, wherein the surgical aid is at least one marker field.

19. The method of claim 15, wherein the surgical aid is at least one locator configured for the intra-operative determination of the position of an implant.

20. The method of claim 15, further comprising;
disengaging the clamp from the bone;
repositioning the clamp at another location on the bone; and
re-anchoring the clamp to the bone.
